(19) United States
(12) Patent Application Publication
(10) Pub. No.: US 2025/0325219 A1
(43) Pub. Date: Oct. 23, 2025

(54) Dynamic Posturography Apparatus with Tunable Optics

(71) Applicant: Natus Acquisition II, LLC, Schaumburg, IL (US)

(72) Inventor: Yan Zhou, Pleasanton, CA (US)

(73) Assignee: Natus Acquisition II, LLC, Schaumburg, IL (US)

(21) Appl. No.: 18/638,018

(22) Filed: Apr. 17, 2024

(51) Int. Cl. A61B 5/00; G02B 26/00; G02C 7/08; G02F 1/1334; G02F 1/137; G02F 1/163

(52) U.S. Cl. CPC A61B 5/4023; A61B 5/6803; A61B 5/742; G02B 26/004; G02C 7/081; G02F 1/1334; G02F 1/137; G02F 1/163; A61B 2562/0219; G02F 2201/44

(57) ABSTRACT

A posturography apparatus including a force plate, a display device, a goggle device that includes a tunable lens operable to transition between a first configuration configured to pass light therethrough with a first refraction and a second configuration configured to pass light therethrough with a second refraction and a lens transition device operable to transition the lens between the first and second configurations. The apparatus further includes a computerized device that includes a display adapter to display information on the display device and a configured to operate the display adapter to display instructions on the display device, operate the lens transition device to change the configuration of the lens of the goggle device, receive ground reaction force measurements from the force plate, and calculate a balance component of the patient from the ground reaction force measurements.

### FIELD OF THE INVENTION

The present invention relates to systems and methods for performing dynamic posturography utilizing tunable optics.

### BACKGROUND OF THE INVENTION

Posturography apparatuses have traditionally required three components: a goggle device worn by the patient, a force plate for measuring ground reactive forces when the user stands upon it, and a large rotatable “box” having a simulated scene depicted for the user to observe. The box is then rotated, such that one or both of the user and the depicted scene rotate, creating a discordance between the user's visually perceived rotational orientation and their perceived rotational orientation through the body's other posture systems. A disadvantage of this type of system is it requires substantial machinery to accomplish this type of rotation, increasing the weight of the system, reducing the portability of the system, and introducing mechanical parts that are subject to an increased failure rate. Accordingly, there is a need in the art for a posturography analysis apparatus that does not rely on a “box” to create the mismatch between the user's perceived visual rotational orientation and their rotational orientation as perceived through other body posture systems.

### SUMMARY OF THE INVENTION

With the above in mind, embodiments of the present invention are directed to a posturography apparatus with tunable optics. A posturography apparatus according to an embodiment of the invention comprises a force plate operable to measure ground reaction forces resulting from a patient standing on the upper surface thereof, a display device positionable to be viewable by the patient, and a goggle device configured to be worn on a head of the patient to overlie eyes of the patient, the goggle device comprising a tunable lens operable to transition between a first configuration configured to pass light therethrough with a first refraction and a second configuration configured to pass light therethrough with a second refraction, and a lens transition device operable to transition the lens between the first configuration and the second configuration. The posturography apparatus further comprises a computerized device that in turn comprises a display adapter operably coupled to the display device and configured to display information on the display device and a controller operably coupled to each of the goggle device, the force plate, and the display adapter. The controller is configured to operate the display adapter to display instructions on the display device, operate the lens transition device to change the configuration of the lens of the goggle device, receive ground reaction force measurements from the force plate, and calculate a balance component of the patient from the ground reaction force measurements.

In some embodiments, the tunable lens may comprise a pair of wedge prisms positioned in optical communication with each other. In the first configuration the wedge prisms may be angularly positioned in an orientation where the bases of the wedge prisms are one of in a first orientation where the bases are at opposite ends of the respective edge prisms, in a second orientation where the bases are adjacent to each other at a lower orientation relative to the goggles, and in a third orientation where the bases are adjacent to each other at an upper orientation relative to the goggles. In the second configuration the wedge prisms may be angularly positioned in one of the first orientation, the second orientation, and the third orientation, where the second configuration is different from the first configuration. The lens transition device may be configured to counter-rotated each wedge prism to change the angular position of the bases thereof.

In some embodiments the tunable lens may be an optical cell comprising optical fluid contained within the optical cell at least one transparent optical element. The lens transition device may be operable to change an angular orientation of the transparent optical element. Changing the angular orientation of the transparent optical element may change a refraction state of the optical cell. In some further embodiments, the transparent optical element may define at least a portion of an end of an outer wall of the optical cell within which the optical fluid is contained. The transparent optical element may be a first transparent optical element and the tunable lens may further comprise a second transparent optical element that defines a portion of an end of the outer wall of the optical cell that is opposite the end of the outer wall that is at least partially defined by the first transparent optical element. The lens transition device may be operable to change the angular orientations of the first and second transparent optical elements in opposing angular directions. In other further embodiments, the optical fluid comprised by the optical cells may comprise a first optical fluid positioned within a first internal region of the optical cell, the first optical fluid having a first refractive index and a second optical fluid positioned within a second internal region of the optical cell, the second optical fluid having a second refractive index. The transparent optical element may be positioned between the first and second optical fluids, preventing any mixing of the first and second optical fluids, and partially defining each of the first and second internal regions. Changing the angular orientation of the transparent optical element may change the geometries of the first and second internal regions. In some further embodiments, the lens transition device may be one of a motorized leadscrew, a voice coil actuator, or a piezo actuator.

In some embodiments the goggle device may further comprise a vision denial device operable to transition between a first state where the vision denial device interferes with the transmission of light from the environment to one or both of the eyes of the patient and a second state where the vision denial device does not substantially interfere with the transmission of light from the environment to the eyes of the patient. The controller may be further configured to selectively switch the vision denial device between the first state and the second state for one or both of the eyes of the patient. The vision denial device may comprise a polymer-dispersed liquid crystal (PDLC) film that is electrically switchable between the first and second states and configured to diffuse environmental light passing through the goggle device in the first state and not to diffuse environmental light passing through the goggle device in the second state. The vision denial device may comprise an electrochromic film (EF) that is electrically switchable between the first and second states and configured to reduce a brightness of environmental light passing through the goggle device in the first state and not substantially affect the brightness of environmental light passing through the goggle device in the second state. In some embodiments, the controller may be configured to switch the PDLC and the EF at least one of independently and together.

In some embodiments the force plate may be further configured to change an angle of orientation of an upper surface thereof upon which the patient stands. In some embodiments the tunable lens may further comprise a transparent displacement member configured to be operable to position the tunable lens in a first vision-correcting configuration for correcting hyperopia of the patient and in a second vision-correcting configuration for correcting myopia of the patient.

Embodiments of the invention may also be directed to a method of administering a posturography examination comprising positioning a patient on an upper surface of a force plate that is operable to measure ground reaction forces resulting from the patient standing on the upper surface, positioning a goggle device on a head of the patient to cover two eyes of the patient, operating a lens transition device comprised by the google device to one of transition a tunable lens comprised by the goggle device between a first configuration and a second configuration and maintain the tunable lens in a current configuration, operating the force plate to one of maintain an angle of orientation of the upper surface and change the angle of orientation of the upper surface, receiving ground reaction force measurements from the force plate, and calculating a balance component of the patient from the ground reaction force measurements received from the force plate. In some embodiments, the method may further comprise operating a vision denial device to interfere with transmission of light from the environment to one or both of the eyes of the patient. In some embodiments, the method may further comprise operating a display device positioned to be viewable by the patient to depict instructions to be viewed by the patient.

### BRIEF DESCRIPTION OF THE DRAWINGS

FIG. 1 is an illustration of a deployment of a posturography system according to an embodiment of the invention, showing the transition of lenses comprised by a goggle device thereof between first and second configurations.

FIG. 2 is a schematic view of a posturography apparatus according to an embodiment of the invention.

FIG. 3 presents three orientations of prisms comprised by lenses of goggles of a posturography apparatus according to an embodiment of the invention.

FIG. 4 is a schematic representation of the posturography apparatus of FIG. 3 with the prisms in the third orientation.

FIG. 5 is a perspective view of a force plate comprised by a posturography apparatus according to an embodiment of the invention.

FIG. 6 is a representative view of the optics of a goggle device comprising a vision denial device according to an embodiment of the invention.

FIGS. 7A-7C depict different methods of changing the angular orientation relative to a patient eye of their optical environment comprised by a posturography apparatus according to an embodiment of the invention.

FIGS. 8A-8C depict a tunable lens comprising an optical cell according to an embodiment of the invention.

FIGS. 9A-9C depict a tunable lens comprising an optical cell according to another embodiment of the invention.

FIGS. 10A-10C depict another tunable lens comprising an optical cell according to another embodiment of the invention.

FIG. 11 depicts a flowchart illustrating a method of performing a posturography analysis according to an embodiment of the invention.

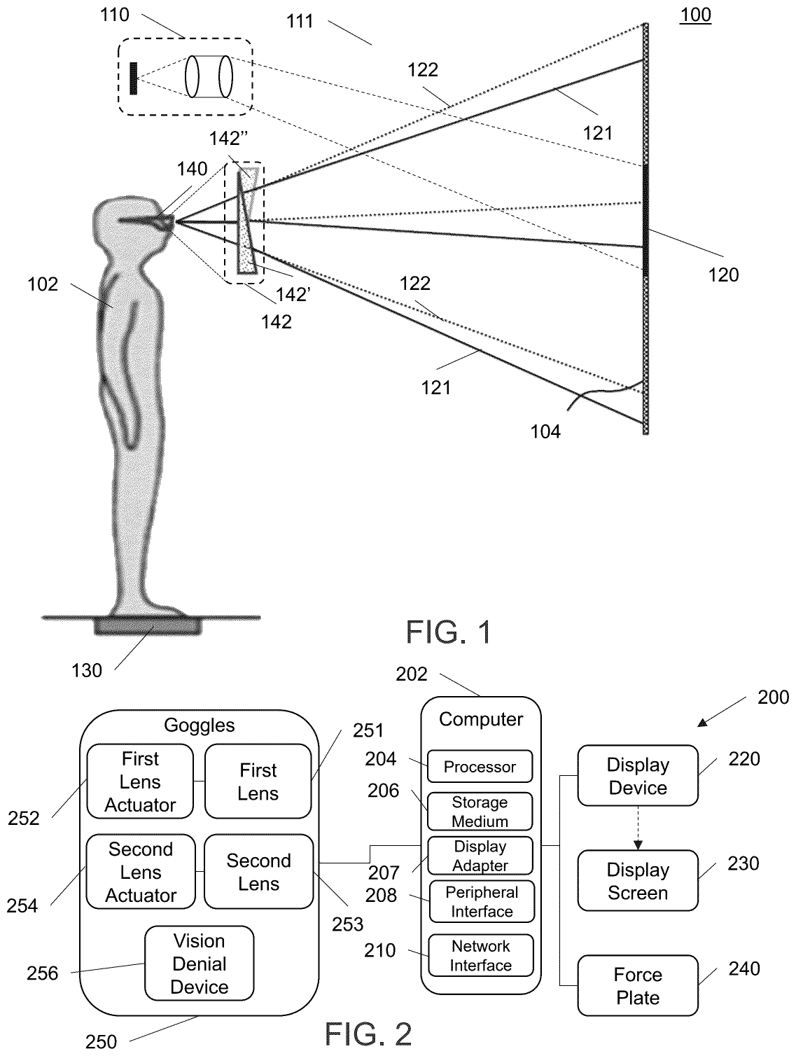

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a computerized dynamic posturography apparatus for assessing the central nervous system adaptive mechanisms involved in the control of posture and balance. The apparatus may be used for diagnosing balance disorders and/or in normal posture screening and evaluation.

A posturography apparatus 100 according to an embodiment of the invention may comprise a display device 110, a display screen 120, a force plate 130, and a goggle device 140. The display device 110 may be any device operable to cause an image or video to be displayed on the display screen 120. The display screen 120 may be positioned so as to be observable/viewable by a user 102. Such image or video being displayed may be accomplished by any means or method known in the art, including projection onto a reflective screen and operation of display devices comprised by the display screen 120, including, but not limited to, liquid crystal display (LCD) devices, light-emitting diode (LED) devices, organic LED (OLED) devices, micro-LED displays, plasma displays, and any other display as is known in the art. The display device 110 and the display screen 120 may be operated to provide instructions for performing a posturography analysis, as will be described in greater detail below. In the present embodiment, a projection 111 from the display device 110 may be incident upon the display screen 120 in a displayed region. In other embodiments, the display screen 120 may be a display device operable to emit light to be perceived by the patient, and the display device 110 may control the operation of the display screen 120.

The goggle device 140 may be worn by the user 102. More particularly, the goggle device 140 may be worn on the head of the user 102 over the eyes of the user 102 such that light from at least a majority of the optical environment that the patient can perceive must pass through the goggle device 140 to be observed by the user 102. The goggle device 140 may comprise one or more lenses 142, as will be described in greater detail below. The one or more lenses 142 may be tunable to refract light passing therethrough. Such refraction may change the perception of the user 102 when using the goggle device 140. The change in perception may be configured to change the perceived bodily orientation of the user 102. Such a change in perception of the bodily orientation of the user 102 may be configured to make the perception of the user 102 such that the user 102 “feels” like they are leaning in a direction (e.g. forwards), thus causing the user 102 to feel the need to lean in an opposite direction (e.g. backwards) to avoid falling over. In the present embodiment, the one or more lenses 142 may be positioned in a first configuration 142′ to provide a first perceived visual orientation stimulus 121, which in this embodiment is perceiving a first visual orientation of at least a majority of the optical environment including the display screen 120. The first perceived visual orientation stimulus may be one in which there is no net refraction by the one or more lenses 142, such that the first perceived visual orientation does not provide any type of mismatch between the physical orientation of the user 102 and the physical orientation suggested by the first perceived visual orientation stimulus 121, or in which there is net refraction by the one or more lenses 142, such that the perceived visual orientation provides a mismatch between the physical orientation of the user 102 and the physical orientation suggested by the first perceived visual orientation stimulus 121. Where there is a net refraction in the first configuration 142′, the direction of the refraction may be in a first direction. The one or more lenses 142 may further be transitioned to a second configuration 142″ to provide a second perceived visual orientation stimulus 122, which in this embodiment is a second visual orientation of at least a majority of the optical environment including the display screen 120 that is different than the first visual orientation including the display screen 120 of the first configuration 142′. The second configuration 142″ may refract light passing therethrough to cause the second perceived visual orientation stimulus 122, changing the perceived physical orientation of the user 102 based on their vision. The refraction may be in any direction, e.g. vertically, horizontally, rotationally, or by any other modality as is known in the art. Where the first configuration 142′ provides a net refraction in the first direction, the refraction in the second configuration 142″ may be in a second direction that is opposite to the first direction and/or perpendicular to the first direction.

While first and second configurations are disclosed, it is contemplated and included within the scope of the invention that any number of configurations of the lenses 142 may be comprised by the invention. One of such additional configurations may be configured to refract light in a direction opposite the direction of refracted light of the second configuration 142″. Another of such additional configurations may refract light at some proportion of the refraction of the second configuration 142″. It is contemplated and included within the scope of the invention that the lenses 142 may be transitionable to any number of configurations configured to refract light in any direction and at any magnitude as such permutations are possible.

As the perceived visual orientation stimulus of the user 102 changes, the user 102 will responsively control their body through engagement of various muscles to maintain their postural equilibrium. Such responses can be measured by the force plate 130. The force plate 130 may be a device upon which the user 102 stands and measures ground reactive forces exerted thereupon by the user 102. By coordinating the measured ground reactive forces with the changes in the perceived visual orientation stimulus experienced by the user 102, the posturography analysis of the user 102 may be accomplished. The force plate 130 may comprise a surface upon which the user 102 may stand and one or more force sensors (not shown) configured to measure the ground reactive forces as described above and as is known in the art.

Referring now to FIG. 2, a posturography apparatus 200 according to an embodiment of the invention is presented. The posturography apparatus 200 comprises a computer device 202. The computer device 202 may be any computerized device as is known in the art, including personal computers, tablet devices, servers, and the like. The computer device 202 may comprise hardware components necessary for operation of computerized devices, including a processor 204, a non-transitory computer-readable medium 206, a display adapter 207, a peripheral device interface device 208, and a network interface device 210. The processor 204 may be any processing device as is known in the art, including, but not limited to, integrated circuits, including microprocessors, field programmable gate arrays, and the like. The non-transitory computer-readable storage medium 206 may be any medium on which software that may be executed by the processor 204 may be stored. Types of media include, but are not limited to, solid-state drives, hard disk drives, flash memory, PROM devices, EPROM devices, EEPROM devices, memristor devices, and any other memory storage devices as are known in the art.

The processor 204 may be configured to operate the display device 220, the force plate 240, and the goggles 250 to conduct a posturography analysis. Additionally, the storage medium 206 may have stored thereupon software that is executable by the processor 204 to conduct a posturography analysis. Additional details about how such a posturography analysis may be conducted by the posturography apparatus 200 will be discussed below.

The display adapter 207 may be configured to connect to a display device, such as a display device 220 comprised by the apparatus 200, and transmit images and/or video to be displayed by the display device 220. The processor 204 may be configured to cause generation and transmission of such images and video to the display device 220 via the display adapter 207. The display adapter 207 may be any type of display device as is known in the art, including graphical processing units, and may further be configured to both perform graphics processing and transmission functions. Such transmission may be wired or wireless. The types of images and videos may comprise instructions for the patient as the posturography analysis is being performed.

The peripheral interface device 208 may be any device configured to connect peripheral devices to the computer 202, including wired and wireless communication devices, such as a universal serial bus (USB) interface device. The processor 204 may be configured to communicate with peripheral devices via the peripheral device interface device, including user input devices such as computer mice, keyboards, and touchscreen devices. In some embodiments, a force plate 240 comprised by the apparatus 200 may be connected to the computer 202 via the peripheral interface 208. In some embodiments, the goggles 250 comprised by the apparatus 200 may be connected to the computer 202 via the peripheral interface 208.

The network interface device 210 may be any interface device operable to communicate across a network with another computerized device. Such communication may be wired or wireless. Types of network interface devices include, but are not limited to, Ethernet devices, IEEE 802.xx-compliant devices, including Wi-Fi, Bluetooth, Zigbee, Z-wave, and Matter devices, and cellular communication devices including 3G, 4G, 5G, and 6G devices. The processor 204 may be configured to communicate with remote computerized devices via the network interface device across one or more networks, including personal area networks, local area networks, and wide area networks, including the Internet.

The display device 220 may be any type of display device as mentioned above. The display device 220 may be positioned in communication with the computer 202, more specifically with the display adapter 207, and cause the display of instructions to be observed and performed by a user/patient in performance of a posturography evaluation. The display device 220 may cause the display of images and/or video on a display screen 230, which may be observable by the user/patient. The force plate 240 may be a device for measuring ground reactive forces as described above. The force plate 240 may be positioned in communication with the computer 202, more specifically with at least one of the peripheral interface 208 or the network interface 210.

The apparatus 200 may further comprise a pair of goggles 250. As mentioned above, the goggles 250 may comprise one or more lenses that are configured to transition between first and second configurations. In the first configuration, the lenses may permit light to pass therethrough with zero or negligible net refraction. In the second configuration, the lenses may be configured to refract light passing therethrough. Such refraction may be in any direction. In the present direction, the refraction is vertical, thereby causing the perceived physical orientation of the user/patient to be rotated in a manner that causes the user/patient to feel they are leaning forward or backward.

The goggles 250 may comprise one or more tunable lenses and one or more tunable lens actuators. In the present embodiment, the goggles 250 comprise a first tunable lens 251, a second tunable lens 253, a first tunable lens actuator 252, and a second tunable lens actuator 254. The first tunable lens actuator 252 may be configured to actuate the first tunable lens 251 between first and second configurations having respective first and second tunable lens profiles as described above, and the second tunable lens actuator 254 may be configured to actuate the second tunable lens 253 between first and second configurations having respective first and second tunable lens profiles as described above. The nature and/or principle of operation of the first and second tunable lens actuators 252, 254 may depend on the structure/principle of operation of the first and second tunable lenses 251, 253. The goggles 250 may further comprise a vision denial device 256 operable to interfere with the transmission of light to the first and second lenses 251, 253 by either reflecting, absorbing, or diffusing light passing through the vision denial device 256. Additional details regarding the vision denial device 256 will be discussed below.

The force plate 240 may transmit the measured ground reactive forces to the processor 204 for analysis. The processor 204 may calculate one or more balance components of the patient responsive to both the position of the first and second tunable lenses 251, 253 and the measured ground reactive forces received from the force plate 240. Balance components include, but are not limited to, center of gravity, center of pressure, and the like. The processor 204 may be configured to correlate the lens positions and the measured ground reactive forces in time in calculating the patient's center of gravity. The presence and/or activation of the vision denial device 256, more specifically whether the vision denial device 256 is interfering with the patient's visual perception, may be one of controlled by the processor 204 or indicated to the computer 202 via a user interface device (not shown) which may be any type of user interface device as is known in the art, including, but not limited to, touchscreen devices, mice, keyboards, and the like.

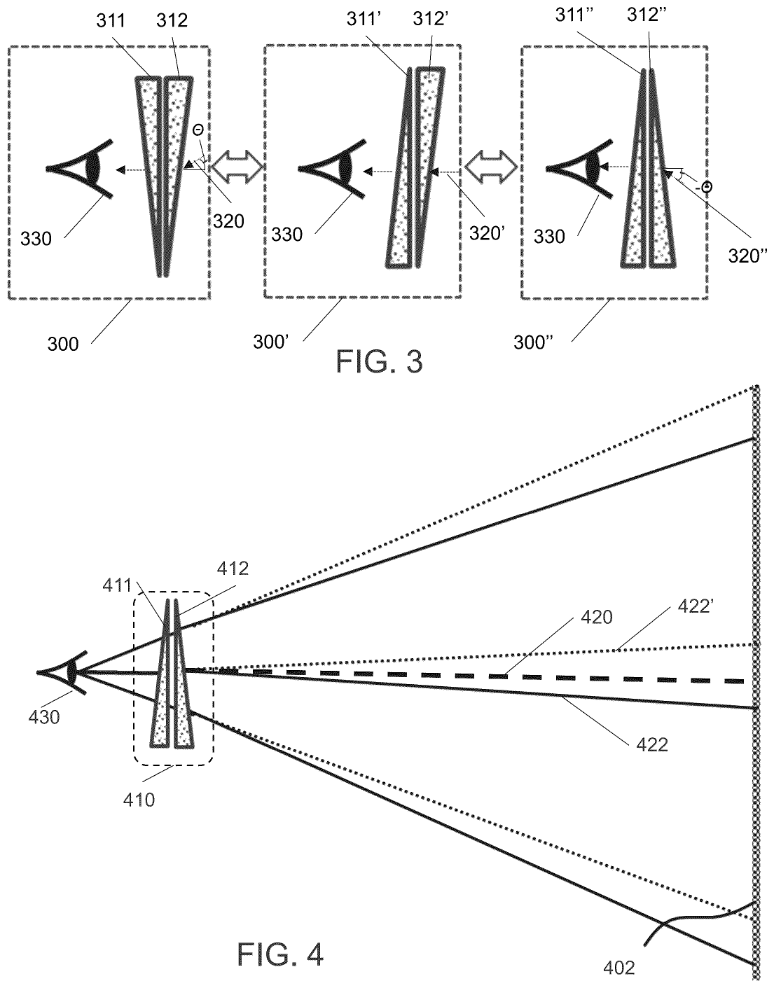

In one embodiment, the first and second tunable lenses 251, 253 may comprise two prisms. An illustration of this embodiments is shown in FIGS. 3-4. As can be seen in orientation 300 of FIG. 3, which is a side view, first and second prisms 311, 312 may be optical wedge structures having an angled surface and a flat surface. The prisms 311, 312 may be formed of optically transparent and refractive materials as are known in the art, including, but not limited to, glass, polycarbonate, plastic, polymer, crystalline structures, and the like. In orientation 300, the first and second prisms 311, 312 may be oriented to have an upward refraction of light passing therethrough. Moreover, because the refractive orientations of the first and second prisms 311, 312 are in the same direction, light being refracted through both the first and second prisms 311, 312 will have a net refraction that is equal to or approximately equal to the sum of the refraction of each of the prisms. Image path 320, which may be understood as a central light ray coming from the perceivable optical environment including the display screen 230, may enter the second prism 312 at angle Θ and exit the first prism 311 at an angle that is approximately parallel to a vertical level of the patient eye 330. Accordingly, the image path 320 as it is incident on an eye of the patient 330 will appear to be vertically refracted relative to the patient eye 330 while in reality the source of the light ray traveling along image path 320 will be above the vertical level. Rotation and/or counter-rotation of the first and second prisms 311, 312 may change the refraction of light passing therethrough, thereby rendering the lens tunable.

In orientation 300′, the first prism 311′ and the second prism 312′ are counter-rotated 90 degrees to cancel the vertical refractive effect thereof as shown in orientation 300. In orientation 300′ which is a top view rather than a side view, the refraction of the first and second prisms 311′, 312′ are oriented in opposite directions, such that their refractive effects oppose each other. In some embodiments, where the degree of refraction of the first and second prisms 311′, 312′ are equal, the net refraction of light passing therethrough will be zero or approximately zero. In some embodiments, where the degree of refraction of the first and second prisms are not equal, the net refraction of light passing therethrough may be non-zero, or even sideways. The central light ray represented by the image path 320′ may enter the second prism 312′ approximately parallel to a vertical level of the patient eye 330 and emit from the first prism 311′ at the same or approximately the same angle, such that there is no or close to no net refraction and the image path 320′ remains approximately parallel to a vertical level of the patient eye 330. Accordingly, in orientation 300′, what appears to the patient to be on a vertical level will in reality be on the vertical level.

In orientation 300″, which is a side view, the first and second prisms 311″, 312″ are both rotated 180 degrees from their orientation in orientation 300, such that both now refract light passing therethrough in a downward direction. Such rotation may be a continuation of the counter-rotation of orientation 300′, with each prism being rotated an additional 90 degrees from that angular orientation shown in orientation 300′. Accordingly, light passing therethrough may be refracted such that it may enter the second prism 312″ at angle-O and exit the first prism 311″ at an angle that is approximately parallel to a vertical level of the patient eye 330. Accordingly, the central light ray represented by the image path 320 as it is incident on an eye of the patient 330 will appear to be vertically level with the patient eye 330 while in reality the source of the light traveling along image path 320 will be below the vertical level.

In such embodiments, the first and second prisms 311, 312 may be independently rotatable respective to each other. Any means or method of rotating optical elements as is known in the art are contemplated and included within the scope of the invention, including, but not limited to, stepper motors, motorized leadscrews, voice coil actuators, and piezo actuators. Such devices are contemplated and included within the scope of the first and second tunable lens actuators 252, 254 of apparatus 200. Moreover, it is contemplated and included within the scope of the invention that each of the first and second tunable lenses 251, 253 may each comprise first and second prisms 311, 312 as described herein.

An orientation analogous to orientation 300″ is shown in FIG. 4, with the lens 410 comprising first and second prisms 411, 412 both in an orientation to refract light downward, such that a central light ray represented by the imaging path 422 that appears to be a vertical level 420 to the patient eye 430 may in actuality be below the vertical level 420. The refraction of light for prisms being in an orientation analogous to orientation 300 of FIG. 3 would be in an upward direction, such that a central light ray represented by the imaging path 422′ that appears to be at the vertical level 420 to the patient eye 430 may in actuality be above the vertical level 420.

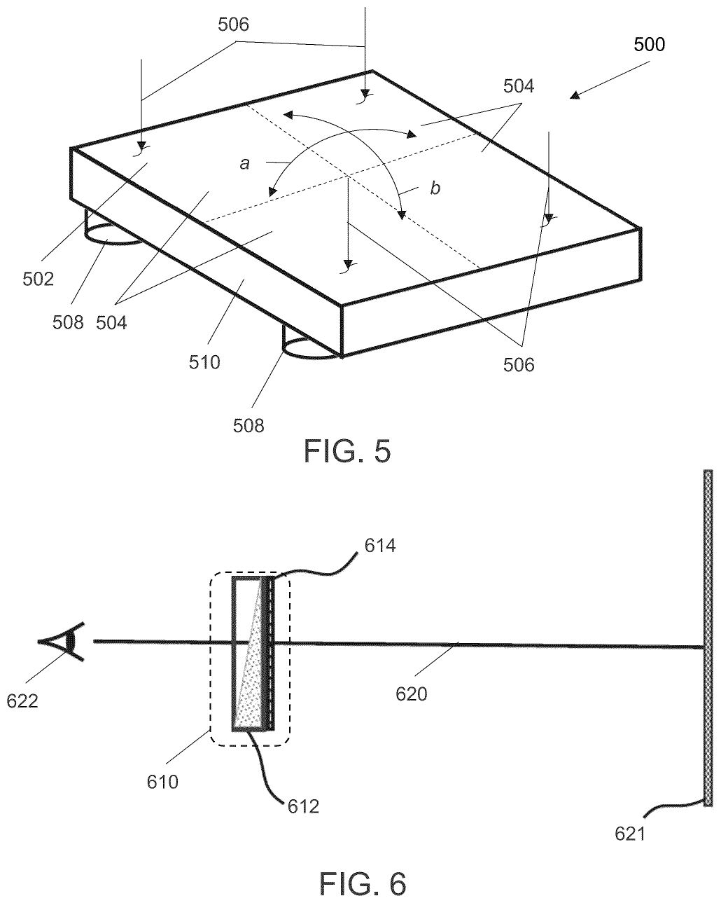

Referring now to FIG. 5, a force plate 500 that may be comprised by a posturography apparatus according to an embodiment of the invention is presented. The force plate 500 may be configured to measure the downward force exerted upon the force plate 500 with the patient standing on an upper surface 502 thereof. The measurement of forces may be accomplished in any way as is known in the art. In some embodiments, force measuring devices may be positioned in four quadrants 504 of the force plate 500 and measure the downward forces 506 exerted in each of those quadrants 504. Such measurements may be provided to a computerized device, as is described hereinabove.

In some embodiments, the force plate 500 may be operable in one of two or more modes, including a stable mode and a sway-referenced mode. In the stable mode, one or more feet 508 of the force plate 500 that may extend downward from a body member 510 of the force plate 500, in some embodiments one foot 508 extending down from an area of the body member 510 corresponding to a quadrant 504, and be configured to interface with a ground surface of the environment may be in a locked configuration such that there is no relative movement between the body member 510 and any of the feet 508. In the stable mode, the upper surface 502 of the force plate 500 may define a plane that is at least one of parallel to a plane defined by the ground surface and orthogonal to the direction of the force of gravity. In the sway-referenced mode, the body member 510 may be permitted to move relative to one or more of the feet 508, rotating about axes a and/or b. Such rotation may result in the plane defined by the upper surface 502 being at least one of skew to the plane defined by the ground surface and not orthogonal to the direction of the force of gravity. In some embodiments, the force plate 500 may be configured to actively change the plane of the upper surface to enforce a desired plane angle.

In some embodiments, goggles comprised by a posturography apparatus according to an embodiment of the invention may comprise a vision denial device. Such a vision denial device may be operable to at least one of completely block all light from being observable by the patient or diffuse or otherwise render light passing therethrough such that the patient cannot perceive recognizable objects or the environment surrounding the patient. Accordingly, the vision denial element may prevent the patient's vision-based aspects of their posture from responding to visual stimuli. An embodiment of such a vision denial element is presented in FIG. 6. A lens 610 of a goggle comprised by a posturography apparatus is presented. The lens 610 may comprise a tunable optic 612 and a vision denial device 614. The vision denial device 614 may be positionable along a light path 620 coming from a display screen 621 so as to obstruct light from passing through the tunable optic 612 and reaching the patient eye 622 in a condition that the patient can discern what is in front of them, by either diffusing or blocking the light along the light path 620. In some embodiments, the vision denial device 614 may be a removable opaque and/or diffusive structure that can be selectively placed in or on the goggles by an operator of the posturography apparatus to interfere with the perception of light. In some embodiments, the vision denial device 614 may be a device that is switchable between two configurations, where in a first configuration light may pass therethrough completely or nearly completely unaffected, such that any diffusion or absorption is sufficiently low that it does not interfere with the patient's ability to perceive what is in front of them, thereby enabling the vision-based aspects of their balance/posture to be affected. In the second configuration, the vision denial device 614 may diffuse or absorb light to prevent the visual effect to the patient's balance/posture. Types of such devices include, but are not limited to, polymer-dispersed liquid crystal (PDLC) devices, such as a PDLC film applied to the tunable lens 612, dye molecule-infused LC devices, and electrochromic films. In some embodiments a PDLC film and an electrochromic film may be used in conjunction with each other to both diffuse and reduce the brightness of light passing therethrough.

Referring back to FIG. 2, the processor 204 may be configured to operate the posturography apparatus 200 to perform a posturography analysis. Such an analysis may comprise various combinations of conditions of various elements of the posturography apparatus. The conditions comprised by the combinations may include the vision denial device being alternated between first and second states where the patient is alternately able to see what is in front of them and having their vision denied, thus being unable to clearly see what is in front of them. The conditions may further include the force plate being in a stable, locked configuration where the upper surface thereof is prevented from moving relative to the ground surface upon which the force plate is positioned and being in a sway-referenced, unlocked configuration where the upper surface may tilt or rotate as described above. The conditions may further include varying at least one of what is displayed on the display screen of the posturography apparatus and the angular orientation of the optical environment including the display screen.

Returning to conducting the posturography analysis, a patient may have their posture/balance tested according to the combination of conditions presented in Table 1.

TABLE 1

| Condition [symbol] | Vision [symbol] | Ground•Surface [symbol] | Visual•Surround [symbol] |
|---|---|---|---|
| 1 [symbol] | Eyes•Open [symbol] | Stable [symbol] | Stable [symbol] |
| 2 [symbol] | Eyes•Closed [symbol] | Stable [symbol] | Stable [symbol] |
| 3 [symbol] | Eyes•Open [symbol] | Stable [symbol] | Sway-Referenced [symbol] |
| 4 [symbol] | Eyes•Open [symbol] | Sway-Referenced [symbol] | Stable [symbol] |
| 5 [symbol] | Eyes•Closed [symbol] | Sway-Referenced [symbol] | Stable [symbol] |
| 6 [symbol] | Eyes•Open [symbol] | Sway-Referenced [symbol] | Sway-Referenced [symbol] |

The analysis may be performed by putting the patient in the conditions in the sequence presented in Table 1, or in a different sequence. Additionally, it is noted that not all possible permutations are presented in Table 1. One embodiment of the invention is directed to the particular conditions presented in Table 1 and excluding other possible combinations of conditions. Other embodiments may include one or more possible combinations in addition to the conditions presented in Table 1. Other embodiments may exclude one or more of the conditions presented in Table 1. Other embodiments may include one or more combination of conditions not shown in Table 1 and exclude one or more conditions shown in Table 1.

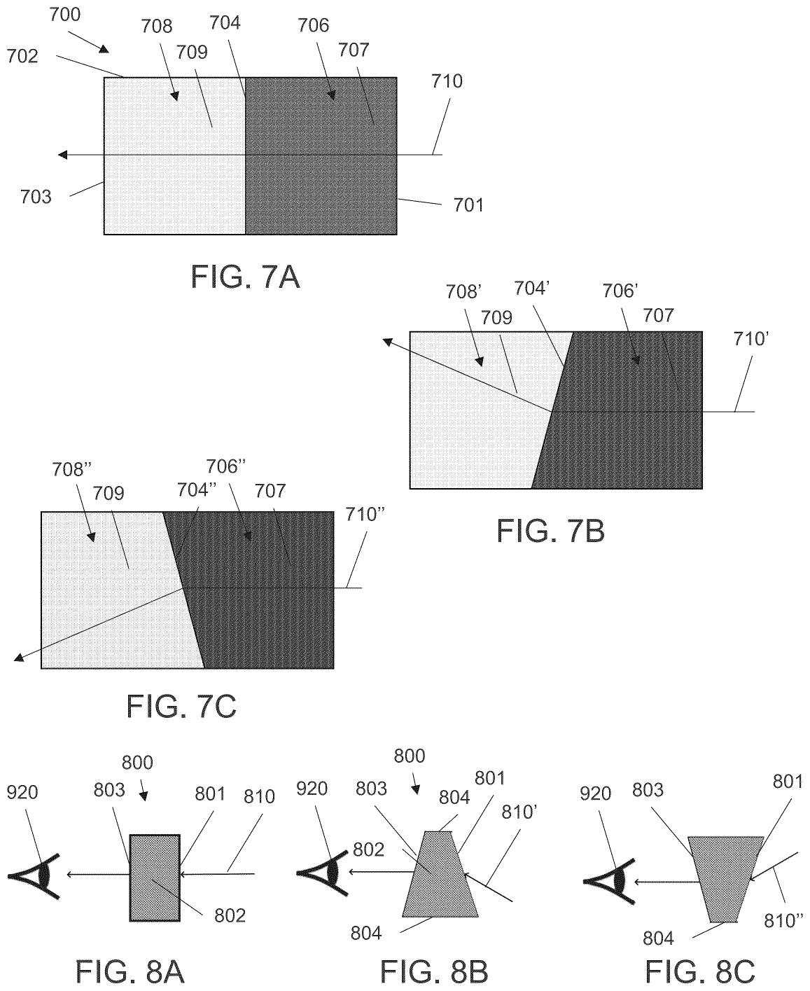

Referring now to FIGS. 7A-7C, a tunable optic 700 according to an embodiment of the invention is presented. The tunable optic 700 may comprise a housing 702 defining an interior space and a segmenting member 704. The housing 702 may comprise first and second transparent optical elements 701, 703 positioned at opposite ends of the lens 700 and configured to permit light to pass therethrough with minimum or almost no reflection, refraction, or absorption. The first and second optical elements 701, 703 may define at least a portion of the respective ends of the optical cell. The segmenting member 704 may be moved between multiple positions to change the geometry of the interior space of the housing 702. The segmenting member 704 may generally segment the interior space of the housing 702 into a first internal region 706 and a second internal region 708. The segmenting member 704 may interface with or be connected to the housing 702 such that it establishes fluidic separation between the first and second internal regions 706, 708. The combination of the housing 702, segmenting member 704, and the first and second transparent optical elements 701, 703 may combine to define an optical cell. The segmenting member 704 may be formed of a transparent material permitting light to pass therethrough. The first internal region 706 may be filled with a first optical fluid 707. The first optical fluid 707 may have a first refractive index. The second internal region 708 may be filled with a second optical fluid 709. The second optical fluid 709 may have a second refractive index. The second refractive index may be greater than, less than, or equal to the first optical index. In embodiments where the second refractive index is equal to the first optical index, the second optical fluid 709 may be the same as the first optical fluid 707.

The segmenting member 704 may be rotatable to change the geometries of the first and second regions. Such rotation may result in a change in the refraction of light passing through the tunable optic 700. In FIG. 7A, the segmenting member 704 is in a first angular orientation that is substantially vertical, such that light travelling along a light path 710 may be unrefracted or not perceptibly refracted when passing through the tunable optic 700. In FIG. 7B, the segmenting member 704′ has been rotated into a second angular orientation that is different from the first angular orientation. In the second angular orientation, the geometries of the first and second internal regions 706′, 708′ may be changed from when in the first angular orientation. Volumes of the first and second internal regions 706′ 708′ in the second angular orientation may be unchanged from the volumes in the first angular orientation, such that neither the first nor second optical fluids 707, 709 are under fluidic compression. In the second angular orientation, light travelling along the light path 710′ may be refracted responsive to at least one of the angle of the segmenting member 704′ and the refractive indices and/or the difference therebetween of the first and second refraction indices. In FIG. 7C, the segmenting member 704″ has been rotated into a third angular orientation that is different from each of the first and second angular orientations. The resulting change in geometry of the first and second internal regions 706″, 708″ is in a general opposite direction from the first and second internal regions 706′, 708′ of the second angular rotation relative to the first and second internal regions 706, 708 of the first angular orientation. As the third angular orientation reflects a rotation in a direction from the first angular orientation that is opposite the rotation direction of the second angular orientation, the direction of refraction of the light path 710″ passing therethrough is also in an opposite direction. The segmenting member 704 may be transitioned between angular orientations by any means or method as is known in the art, including those described above.

In an alternative embodiment, a tunable lens 700 may comprise first and second transparent optical elements 701, 703 as described above, but no segmenting member 704. In such an embodiment, the lens transition device may be configured to change the angular orientation of one or both of the first and second transparent optical elements 701, 703. Such change in angular orientation may refract light passing therethrough in a manner consistent with the types of refraction as described above. Such an embodiment is present in FIGS. 8A-C. In FIG. 8A, a lens 800 having first and second optical elements 801, 803 are presented in a first orientation where a central light ray 810 travelling along a light path may pass through the first and second optical elements 801, 803 having no or nearly no refraction, thereby permitting the central light ray 810 to continue on the same or essentially the same light path to the patient eye 920. An optical fluid 802 as described above may be contained within the lens 800 and occupy the space between the first and second optical elements 801, 803, facilitating the transmission of light therebetween.

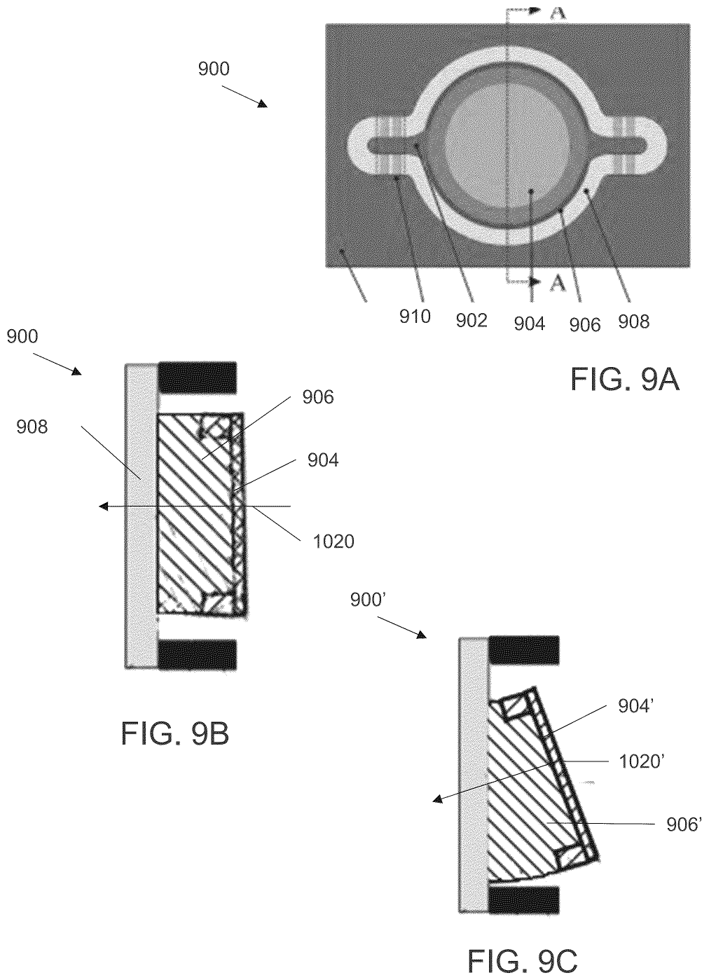

In FIG. 8B, the first and second optics 801, 803 have been counter-rotated to second configurations whereby the central light ray 810′ is now refracted by each of the optics 801, 803 as it approaches the patient eye 920. Such counter-rotation may be accomplished by any rotational means, mechanism, or method as described above. To accommodate the change in geometry of the lens 800 resulting from the counter-rotation of the first and second optics 801, 803, the lens 800 may comprise an elastic sidewall 804 that allow the change in shape of the lens 800, as shown in the change from FIG. 9B with the lens 800 having a rectangular profile when viewed from the side to FIG. 9C with the lens having a trapezoidal profile when viewed from the side. The sidewall 804 may be attached to the first and second optics 801, 803 and cooperate therewith in defining the cavity within which the optical fluid 802 is contained.

In FIG. 8C, the first and second optics 801, 803 have been counter-rotated to third configurations whereby the central light ray 810″ is now refracted by each of the optics 801, 803 in a direction opposite the direction of refraction of FIG. 8B as it approaches the patient eye 820.

Referring now to FIGS. 9A-9C, another embodiment of a tunable lens 900 according to an embodiment of the invention is presented. The tunable lens 900 may comprise a frame 902, an outer membrane 904 carried by the frame 902, optical fluid 906, and a transparent optical member 908 upon which the optical fluid 906 is positioned. The frame 902 may be rotated with a pivot point along the center line of the lens by a lens transition device 910 as described hereinabove. Such rotation of the frame may cause the outer membrane 904 to rotate, thereby cause a change in the geometry of the optical fluid 906 while keeping the volume of the fluid constant. Such a change in geometry may change the refractive characteristics of the optical fluid 906, and hence change the refraction characteristics of the tunable lens 900. In some embodiments, the transparent optical member 908 may itself be actuatable to be positioned at an angle that is not the vertical angle shown in FIGS. 9B-9C, such that it can also refract light passing therethrough. In such an embodiment, the lens transition device 910 may be further operable to actuate the transparent optical member 908.

In FIG. 9B, which is a side view, the lens 900 is in a first configuration where light travelling along a light path 920 may be generally unrefracted or not perceptibly refracted. In FIG. 9C, which is a side view, the lens 900′ has been rotated to a second configuration through operation of the lens transition device 910, pivoting along the center line of the lens, thus causing the outer membrane 904′ to rotate, thereby changing the geometry of the optical fluid 906 as described above. Accordingly, light travelling along a light path 920′ will be refracted by the lens 900′.

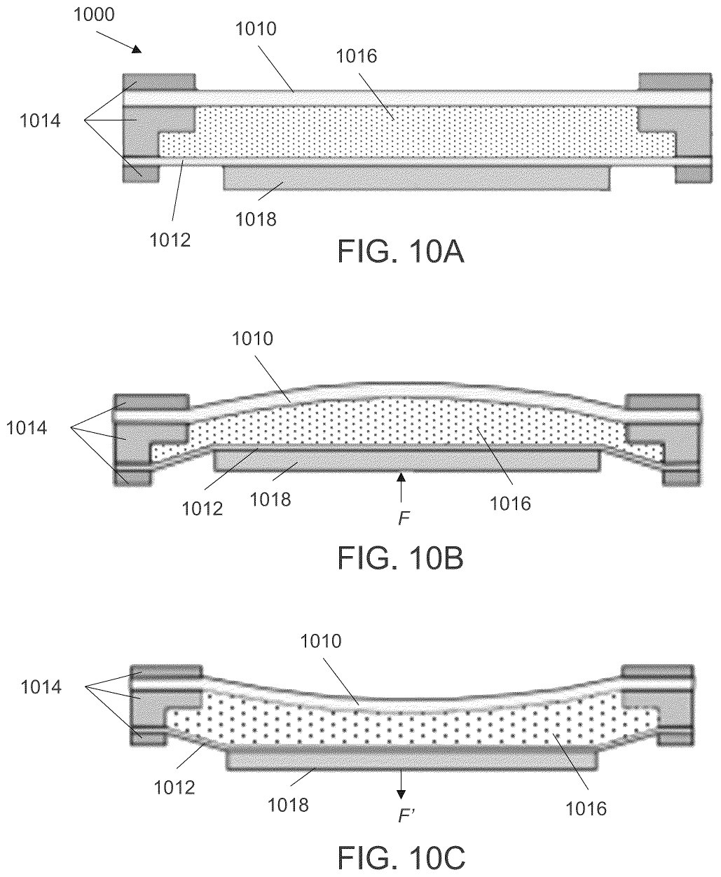

Referring now to FIGS. 10A-C, another aspect of the lenses comprised by the goggles of a posturography apparatus according to an embodiment of the invention is presented. In addition to refracting light generally upwards and/or downwards to affect the visual perception of the posture of the patient, the lens 1000 of the present embodiment may further be operable to transition between configurations for accommodating patients having myopia or hyperopia. For patients having normal vision (neither myopic nor hyperopic), the lens 1000 shown in FIG. 10A in a first configuration may be employed. The lens 1000 may comprise a front transparent elastic member 1010, a rear transparent elastic member 1012, a frame 1014 configured to support the front and rear transparent elastic members 1018, 1012, optical fluid 1016 contained within the frame 1014 and the front and rear transparent elastic members 1010, 1012, and a transparent displacement member 1018.

The transparent displacement member 1018 may be operable to displace the rear transparent elastic member 1012, resulting in either compression or expansion of the optical fluid 1016, which may in turn cause the displacement of the front transparent elastic member 1010. Displacement of the front transparent elastic member 1010 may impart either a concave or convex shape thereto, causing the refraction of light to address either myopic or hyperopic vision.

As shown in FIG. 10B a force F may be applied to the transparent displacement member 1018, putting the transparent displacement member in a first vision-correcting configuration. As described above, the rear transparent elastic member 1012 may be displaced by the transparent displacement member 1018, the optical fluid may be compressed, thereby increasing a fluidic pressured exerted by the optical fluid 1016 on the front transparent elastic member 1010, thereby imparting a convex shape thereto. In this configuration, the lens 1000 may facilitate performance of the posturography analysis while compensating for the hyperopia of the patient. The frame 1014 may prevent translation of the lens 1000 in the direction of force F by being attached to the googles as described above, which are in turn attached to the patient's head.

As shown in FIG. 10C a force F′ may be applied to the transparent displacement member 1018. As described above, the rear transparent elastic member 1012 may be displaced by the transparent displacement member 1018, putting the transparent displacement member in a second vision-correcting configuration. The optical fluid may be expanded, reducing the fluidic pressure it exerts on the front transparent elastic member 1012. The differential between the reduced fluidic pressure of the optical fluid 1016 and ambient air pressure may push upon the front transparent elastic member 1010 in the same direction as force F′, thereby imparting a concave shape thereto. In this configuration, the lens 1000 may facilitate performance of the posturography analysis while compensating for the myopia of the patient. It should be noted that the configurations shown in FIGS. 10A-C can be combined with the configurations shown in FIGS. 9A-C or those of FIGS. 8A-C or those of FIGS. 7A-C. Alternatively, a single lens can be designed to simultaneously have the functions of both refractive error correction and up-down rocking of the optical environment visually perceived by the patient.

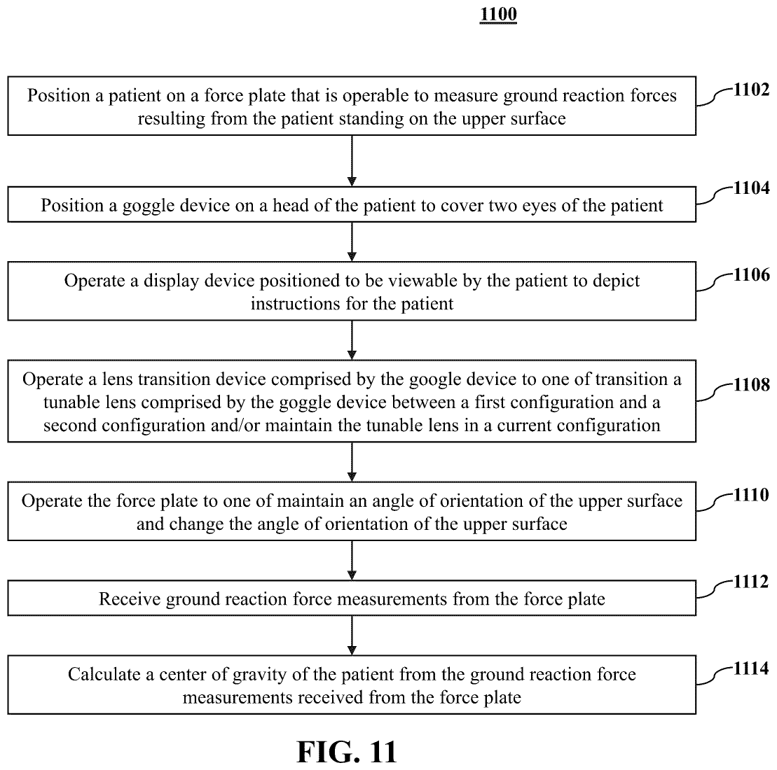

FIG. 11 is a flowchart of an exemplary method 1100 of performing a posturography analysis according to an embodiment of the invention. In some implementations, one or more process blocks of FIG. 11 may be performed by a posturography apparatus. As shown in FIG. 11, method 1100 may include positioning a patient on a force plate that is operable to measure ground reaction forces resulting from the patient standing on the upper surface (block 1102). As also shown in FIG. 11, method 1100 may include positioning a goggle device on the head of the patient to cover the eyes of the patient (block 1104). As further shown in FIG. 11, method 1100 may include operating a display device positioned to be viewable by the patient to depict an image or video to be viewed by the patient (block 1106). Such image or video may be configured to provide instructions to the patient for performing the posturography analysis.

Method 1100 may further include operating a lens transition device, comprised by the goggle device, to transition a tunable lens between a first configuration and a second configuration and/or maintain the tunable lens in a current configuration (block 1108). Such a transition may cause the patient to visually perceive a change in their posture/center of gravity. As further shown in FIG. 11, method 1100 may further include operating the force plate to at least one of maintain an angle of orientation of the upper surface and/or change or permit the change of the angle of orientation of the upper surface (block 1110). As also shown in FIG. 11, method 1100 may include receiving ground reaction force measurements from the force plate (block 1112). Method 1100 may include calculating a center of gravity of the patient from the ground reaction force measurements received from the force plate (block 1114).

Although FIG. 11 shows example blocks of method 1100, in some implementations, method 1100 may include additional blocks, fewer blocks, different blocks, or differently arranged blocks than those depicted in FIG. 11. Additionally or alternatively, two or more of the blocks of method 1100 may be performed in parallel.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced items.

What is claimed is:

1. A posturography apparatus comprising:
a force plate operable to measure ground reaction forces resulting from a patient standing on the upper surface thereof;
a display device positionable to be viewable by the patient;
a goggle device configured to be worn on a head of the patient to overlie eyes of the patient, the goggle device comprising:
a tunable lens operable to transition between:
a first configuration configured to pass light therethrough with a first refraction; and
a second configuration configured to pass light therethrough with a second refraction; and
a lens transition device operable to transition the lens between the first configuration and the second configuration; and
a computerized device comprising:
a display adapter operably coupled to the display device and configured to display information on the display device; and
a controller operably coupled to each of the goggle device, the force plate, and the display adapter, the controller being configured to:
operate the display adapter to display instructions on the display device;
operate the lens transition device to change the configuration of the lens of the goggle device;
receive ground reaction force measurements from the force plate; and
calculate a balance component of the patient from the ground reaction force measurements.

2. The posturography apparatus of claim 1 wherein:
the tunable lens comprises a pair of wedge prisms positioned in optical communication with each other;
in the first configuration the wedge prisms are angularly positioned in an orientation where the bases of the wedge prisms are one of in a first orientation where the bases are at opposite ends of the respective edge prisms, in a second orientation where the bases are adjacent to each other at a lower orientation relative to the goggles, and in a third orientation where the bases are adjacent to each other at an upper orientation relative to the goggles;
in the second configuration the wedge prisms are angularly positioned in one of the first orientation, the second orientation, and the third orientation, where the second configuration is different from the first configuration; and
the lens transition device is configured to counter-rotated each wedge prism to change the angular position of the bases thereof.

3. The posturography apparatus of claim 1 wherein:
the tunable lens is an optical cell comprising:
optical fluid contained within the optical cell; and
at least one transparent optical element;
the lens transition device is operable to change an angular orientation of the transparent optical element; and
changing the angular orientation of the transparent optical element changes a refraction state of the optical cell.

4. The posturography apparatus of claim 3 wherein the transparent optical element defines at least a portion of an end of an outer wall of the optical cell within which the optical fluid is contained.

5. The posturography apparatus of claim 4 wherein:
the transparent optical element is a first transparent optical element;
the tunable lens further comprises a second transparent optical element that defines a portion of an end of the outer wall of the optical cell that is opposite the end of the outer wall that is at least partially defined by the first transparent optical element; and
the lens transition device is operable to change the angular orientations of the first and second transparent optical elements in opposing angular directions.

6. The posturography apparatus of claim 3 wherein:
the optical fluid comprised by the optical cells comprises:
a first optical fluid positioned within a first internal region of the optical cell, the first optical fluid having a first refractive index; and
a second optical fluid positioned within a second internal region of the optical cell, the second optical fluid having a second refractive index;
the transparent optical element is positioned between the first and second optical fluids, preventing any mixing of the first and second optical fluids, and partially defining each of the first and second internal regions; and
changing the angular orientation of the transparent optical element changes the geometries of the first and second internal regions.

7. The posturography apparatus of claim 3 wherein the lens transition device is one of a motorized leadscrew, a voice coil actuator, or a piezo actuator.

8. The posturography apparatus of claim 1 wherein:
the goggle device further comprises a vision denial device operable to transition between a first state where the vision denial device interferes with the transmission of light from the environment to one or both of the eyes of the patient and a second state where the vision denial device does not substantially interfere with the transmission of light from the environment to the eyes of the patient; and
the controller is further configured to selectively switch the vision denial device between the first state and the second state for one or both of the eyes of the patient.

9. The posturography apparatus of claim 8 wherein the vision denial device comprises a polymer-dispersed liquid crystal (PDLC) film that is electrically switchable between the first and second states and configured to diffuse environmental light passing through the goggle device in the first state and not to diffuse environmental light passing through the goggle device in the second state.

10. The posturography apparatus of claim 9 wherein the vision denial device further comprises an electrochromic film (EF) that is electrically switchable between the first and second states and configured to reduce a brightness of environmental light passing through the goggle device in the first state and not substantially affect the brightness of environmental light passing through the goggle device in the second state.

11. The posturography apparatus of claim 10 wherein the controller is configured to switch the PDLC and the EF at least one of independently and together.

12. The posturography apparatus of claim 8 comprises an electrochromic film (EF) that is electrically switchable between the first and second states and configured to reduce a brightness of environmental light passing through the goggle device in the first state and not substantially affect the brightness of environmental light passing through the goggle device in the second state.

13. The posturography apparatus of claim 1 wherein the force plate is further configured to change an angle of orientation of an upper surface thereof upon which the patient stands.

14. The posturography apparatus of claim 1 wherein the tunable lens further comprises a transparent displacement member configured to be operable to position the tunable lens in a first vision-correcting configuration for correcting hyperopia of the patient and in a second vision-correcting configuration for correcting myopia of the patient.

15. A method of administering a posturography examination comprising:
positioning a patient on an upper surface of a force plate that is operable to measure ground reaction forces resulting from the patient standing on the upper surface;
positioning a goggle device on a head of the patient to cover two eyes of the patient;
operating a lens transition device comprised by the google device to one of transition a tunable lens comprised by the goggle device between a first configuration and a second configuration and maintain the tunable lens in a current configuration;
operating the force plate to one of maintain an angle of orientation of the upper surface and change the angle of orientation of the upper surface;
receiving ground reaction force measurements from the force plate; and
calculating a balance component of the patient from the ground reaction force measurements received from the force plate.

16. The method of administering a posturography examination of claim 15 further comprising operating a vision denial device to interfere with transmission of light from the environment to one or both of the eyes of the patient.

17. The method of administering a posturography examination of claim 15 further comprising operating a display device positioned to be viewable by the patient to depict instructions to be viewed by the patient.
